(19) United States
(12) Patent Application Publication
(10) Pub. No.: US 2025/0248833 A1
(43) Pub. Date: Aug. 7, 2025

(54) CONICAL STENT FOR INTRACRANIAL ANGIOPLASTY AND METHODS OF STENT PLACEMENT FOR PREVENTION AND TREATMENT OF ISCHEMIC STROKE

(71) Applicant: DyQure LLC, Sauk Rapids, MN (US)

(72) Inventors: Adnan I. Qureshi, Columbia, MO (US); Muhammad Fareed Khan Suri, Sauk Rapids, MN (US)

(21) Appl. No.: 18/435,747

(22) Filed: Feb. 7, 2024

(51) Int. Cl. A61F 2/958; A61F 2/00; A61F 2/915; A61M 25/10

(52) U.S. Cl. CPC A61F 2/958; A61M 25/104; A61F 2002/0081; A61F 2002/91575; A61F 2230/0067; A61F 2230/0078; A61M 25/1002

(57) ABSTRACT

A balloon expandable stent having multiple rings and cells with ultra-thin struts, wherein the sizes of the cells are larger proximally and varies amongst the rings, enabling the stent to expand to a larger diameter at its proximal end compared with the distal end. A conical shape rather than the classic cylindrical design post deployment ensures adequate opposition of the stent to the arterial wall and avoids supramaximal expansion at the distal end. A unique monorail angioplasty balloon catheter delivers the stent through tortuous intracranial arteries with longer working length. A longer segment with traversing microwire enables modification in stiffness depending on stiffness of microwire, and reduction in length of segment with a hypotube alone.

### TECHNICAL FIELD

The invention relate to devices and methods for prevention and treatment of stroke, and more specifically, apparatuses and methods for treating intracranial arterial diseases that cause or exacerbate ischemic stroke.

### BACKGROUND

Atherosclerotic disease often involves the intracranial arteries including those encased by cranial bones and dura, and those located in the subarachnoid space. Age, hypertension, and diabetes mellitus are independent risk factors for intracranial atherosclerosis. Intracranial atherosclerosis can result in thromboembolism with or without hypoperfusion leading to transient or permanent cerebral ischemic events.

High rates of recurrent ischemic stroke and other cardiovascular events mandate early diagnosis and treatment. Present treatment is based on a combination of antiplatelet drugs, optimization of blood pressure and LDL cholesterol values, and intracranial angioplasty or stent placement, or both, in selected patients. Intracranial angioplasty and stent placement are used for patients with high grade (70-99%) stenosis of a major intracranial artery such as the vertebral or basilar arteries, and internal carotid and middle cerebral arteries with recurrent ischemic symptoms. Additional criteria such as the occurrence of ischemic symptoms despite the use of antithrombotic treatment or severe hypoperfusion by regional blood flow studies are often used.

Renewed focus has been placed on balloon expandable stents for intracranial stenosis, after self-expanding stents were found to be inferior to best medical treatment in a study entitled Stenting vs. Aggressive Medical Management for Preventing Recurrent Stroke in Intracranial Stenosis (SAMMPRIS). See, Chimowitz M I, Lynn M J, Derdeyn C P, et al. Stenting Versus Aggressive Medical Therapy for Intracranial Arterial Stenosis. N Engl J Med. 2011; 365:993-1003. The 1-year rate of the primary endpoint (any stroke or death within 30 days or ipsilateral stroke after 30 days) was significantly higher in the self-expanding stent treatment group than in the medically managed group (20% vs 12%).

One of the limitations of self-expanding stents is the higher stenosis rate of 25-30% within 6 months to a year after the procedure. The most important reason is stent design. A self-expendable stent (Wingspan Stent System with the Gateway PTA Balloon Catheter) has been used in many trials, and is delivered through a long and inflexible catheter requiring considerable effort to get the device through the tortuous intracranial arteries. Such mechanical strain and rigorous pushing through the tortuous intracranial arteries results in damage to arteries of the brain causing procedure related strokes. When the device reached the point of stenosis, the self-expanding nitinol stent did not have enough radial force to increase the lumen of the intracranial arteries and stenosis persisted.

Balloon expandable stents that were developed for coronary (heart) blood vessels have been tried for treatment of intracranial stenosis. Balloon expandable stents are expected to increase the durability of the treatment by increasing the lumen diameter more than primary angioplasty or self-expanding stents. A previous comparison demonstrated that post-procedural residual stenosis was significantly lower in patients treated with balloon expandable stents compared with primary angioplasty (4.1% versus 15.1%, p=0.09). These stents were made for coronary arteries which are relatively straight and the inflexible delivery system was unable to move through the tortuous intracranial arteries. The mechanical strain and rigorous pushing through the tortuous intracranial arteries resulted in damage to arteries of the brain causing procedure related strokes. The stents were stiff and suited for the thick coronary arteries but caused injury to the arterial wall of much thinner intracranial arteries.

Inability to deliver the stent to a target lesion, however, has been identified as the main reason for limited value of balloon expandable stents for intracranial stenosis. The limited trackability of balloon expandable stents through tortuous intracranial circulation remains a major limitation and improvement in trackability would be expected to increase the technical success of the procedure.

Therefore, what is needed are devices and methods that increase the trackability of balloon mounted stents in the intracranial circulation and provide an effective method to reduce the high risk of death or disability associated with intracranial atherosclerotic disease.

### SUMMARY

The present invention includes improved stents, and devices and methods for deployment of the stents, that reduce the high risk of death or disability associated with intracranial atherosclerotic disease. According to an embodiment, a balloon expandable stent has ultra-thin struts of a unique configuration that allow deployment at different diameters along the longitudinal length of the stent. The stent is developed to adapt to intracranial arterial dimensions where the segment of artery distal to stenosis is smaller than the segment proximal to the stenosis. The sizes of the cells are larger proximally in an open cell design which enables the stent to expand to a larger diameter at its proximal end compared with its distal end.

The increase in diameter can be gradual with each ring having larger cells with the proximal-most ring having the largest cells. The overall post-deployment conical configuration of the stent, rather than the classic cylindrical configuration, ensures adequate biasing of the stent toward the arterial wall and avoids supramaximal expansion at the distal end.

In an embodiment, the above-mentioned stent is mounted on a balloon asymmetrically, with the distance between the proximal end of the stent and the proximal end of the balloon 2-3 times greater than the distance between the distal end of the stent and the distal end of the balloon. The larger proportion of the balloon that is not constrained by the overlying stent enables the initial inflation of the balloon to be larger in the proximal segment to facilitate a larger inflation in the proximal segment.

In an embodiment, a conical balloon to deploy the stent has a larger proximal inflation diameter of the balloon resulting in a larger diameter in the proximal segment of the deployed stent.

In embodiments, a unique monorail angioplasty balloon catheter delivers the stent through tortuous intracranial arteries with longer working length. A longer segment with traversing microwire enables modification in stiffness depending on the stiffness of microwire, and reduction in the length of segment of hypotube alone.

In an embodiment, an open cell intracranial stent includes a plurality of rings arranged sequentially in a longitudinal direction. Each ring has a plurality of cells arranged adjacently to each other in a transverse direction generally perpendicular to the longitudinal direction. Each cell includes a pair of struts, each strut in the cell having a first end and an opposing second end, the first ends of the pair of struts joined at a first peak, the second end of each of the pair of struts joined to a second end of one of the pair of struts in a separate, adjacent, one of the plurality of cells in the ring to form a pair of peaks opposing the first peaks. The struts and peaks in the ring form a generally sinusoidal configuration, and each of the plurality of rings is joined to another one of the plurality of rings at a nodal connection. Each of the pair of struts in each one of the plurality of cells in each ring has a uniform strut length dimension. The stent has a pair of opposing ends and the plurality of rings includes a first ring at one of the opposing ends, a second ring, a third ring, a fourth ring, a fifth ring, and a sixth ring at the other of the opposing ends. The uniform strut length dimension of the second ring cells has a relative value of 0.9 times the uniform strut length dimension of the cells of the first ring, the third ring cells have a relative value of 0.85 times the uniform strut length dimension of the cells of the first ring, the fourth ring cells have a relative value of 0.85 times the uniform strut length dimension of the cells of the first ring, the fifth ring cells have a relative value of 1.15 times the uniform strut length dimension of the cells of the first ring, and the sixth ring cells have a relative value of 1.25 times the uniform strut length dimension of the cells of the first ring.

In embodiments, each of the struts is coated with a layer comprising a mixture of nanoparticles of an anti-thrombotic medication and a bioabsorbable polymer. The anti-thrombotic medication can be Tirofiban and the bioabsorbable polymer can be polyanhydride.

In embodiments, the stent defines a plurality of microholes, the microholes filled with a radiopaque substance. The radiopaque substance can be Tantalum or Tantalum Oxide.

In embodiments, the struts have a thickness of from about 40 microns to about 60 microns.

In embodiments, a width at the first peak and a width at each of the second peaks is less than a width of the struts. Further, the stent can form a conical or hyperboloid shape when expanded.

In embodiments, a monorail angioplasty balloon catheter includes a first segment having a first hypotube tube shaft defining a first lumen, a second segment operably coupled to the first segment and having a first body portion defining a second lumen fluidly coupled to the first lumen, and a tapered mandrel, and a third segment operably coupled to the second segment and including a second body portion, a balloon, and a tip portion. The body portion defines a third lumen fluidly coupling the first and second lumen and the balloon, and a fourth lumen. The fourth lumen is adapted to receive a microwire therethrough and extends from an end of the tip portion to a fenestration extending through an outer wall of the body portion adjacent the second segment. A longitudinal length dimension of the third segment can be from about 30 cm to about 40 cm.

In embodiments, the monorail angioplasty balloon catheter has a working length of from about 145 cm to about 155 cm. An interior surface of the fourth lumen can be coated with a hydrophilic coating selected from the group consisting of PVP, polyurethane, polyacrylic acid, polyethylene oxide, polysaccharide and cross-linked polymer.

In embodiments, the tapered mandrel can be coated with PTFE or another polymer. The balloon can be formed from a material selected from the group consisting of thermoplastic polymer, PET, nylon, polyethylene with or without PVC, Pebax®, and polyurethane.

In embodiments, amonorail angioplasty balloon catheter includes a first segment comprising a first hypotube tube shaft defining a first lumen, a second segment operably coupled to the first segment and including a body portion, a balloon, and a tip portion. The body portion defines a second lumen fluidly coupling the first lumen and the balloon, and a third lumen, the third lumen adapted to receive a microwire therethrough and extending from an end of the tip portion to a fenestration extending through an outer wall of the body portion adjacent the first segment, an end of the first segment being tapered adjacent the fenestration. A longitudinal length dimension of the second segment can be from about 30 cm to about 40 cm. A working length of the monorail angioplasty balloon catheter can be from about 145 cm to about 155 cm. The monorail angioplasty balloon catheter can include a tapered mandrel. An interior surface of the third lumen can be coated with a hydrophilic coating selected from the group consisting of PVP, polyurethane, polyacrylic acid, polyethylene oxide, polysaccharide and cross-linked polymer. The balloon can be formed from a material selected from the group consisting of thermoplastic polymer, PET, nylon, polyethylene with or without PVC, Pebax®, and polyurethane.

In embodiments, a method of deploying an intracranial stent in a stenosis in an intracranial artery of a patient, includes providing an intracranial stent having a plurality of rings arranged sequentially in a longitudinal direction, each ring having a plurality of cells arranged adjacently to each other in a transverse direction generally perpendicular to the longitudinal direction, each cell comprising a pair of struts, each strut in the cell having a first end and an opposing second end, the first ends of the pair of struts joined at a first peak, the second end of each of the pair of struts joined to a second end of one of the pair of struts in a separate, adjacent, one of the plurality of cells in the ring to form a pair of peaks opposing the first peaks, such that the struts and peaks in the ring form a generally sinusoidal configuration. Each of the plurality of rings is joined to another one of the plurality of rings at a nodal connection. Each of the pair of struts in each one of the plurality of cells in each ring has a uniform strut length dimension, and the stent has a pair of opposing ends and the plurality of rings includes a first ring at one of the opposing ends, a second ring, a third ring, a fourth ring, a fifth ring, and a sixth ring at the other of the opposing ends. The uniform strut length dimension of the second ring cells has a relative value of 0.9 times the uniform strut length dimension of the cells of the first ring, the third ring cells have a relative value of 0.85 times the uniform strut length dimension of the cells of the first ring, the fourth ring cells have a relative value of 0.85 times the uniform strut length dimension of the cells of the first ring, the fifth ring cells have a relative value of 1.15 times the uniform strut length dimension of the cells of the first ring, and the sixth ring cells have a relative value of 1.25 times the uniform strut length dimension of the cells of the first ring.

The method further includes providing a monorail angioplasty balloon catheter with a first segment comprising a first hypotube tube shaft defining a first lumen, a second segment operably coupled to the first segment and comprising a first body portion defining a second lumen fluidly coupled to the first lumen, and a tapered mandrel, and a third segment operably coupled to the second segment and including a second body portion, a balloon, and a tip portion. The body portion defines a third lumen fluidly coupling the first and second lumen and the balloon, and a fourth lumen. The fourth lumen is adapted to receive a microwire therethrough and extends from an end of the tip portion to a fenestration extending through an outer wall of the body portion adjacent the second segment. A longitudinal length dimension of the third segment is from about 30 cm to about 40 cm.

The method further includes crimping the stent around the balloon of the catheter, introducing the monorail angioplasty balloon catheter into the intracranial artery, positioning the stent across the stenosis, and inflating the balloon to expand the stent. The method can further include steps of deflating the balloon, repositioning the balloon proximally relative to the stent, and reinflating the balloon.

The above summary is not intended to describe each illustrated embodiment or every implementation of the subject matter hereof. The figures and the detailed description that follow more particularly exemplify various embodiments.

### BRIEF DESCRIPTION OF THE DRAWINGS

Subject matter hereof may be more completely understood in consideration of the following detailed description of various embodiments in connection with the accompanying figures, in which:

FIG. 1 is a diagrammatic depiction of an intracranial artery exhibiting a region of stenosis;

FIG. 2 depicts the structure of a stent according to an embodiment of the invention;

FIG. 3A is an elevation view of a cell of a stent according to an embodiment of the invention;

FIG. 3B is an elevation view of a cell of a stent according to another embodiment of the invention;

FIG. 3C is an elevation view of the cell of FIG. 3B in a crimped condition;

FIG. 3D is an elevation view of a stent with the cell of FIG. 3A crimped around a balloon;

FIG. 3E is an elevation view of a stent with the cell of FIG. 3B crimped around a balloon;

FIG. 4 is an elevation view of a cell of a stent according to another embodiment of the invention;

FIG. 5A is an elevation view of a cell of a stent according to another embodiment of the invention;

FIG. 5B is an end view of a stent incorporating the cell of FIG. 5A;

FIG. 6A depicts the structure of a stent according to an embodiment of the invention in a flat initial configuration;

FIG. 6B depicts the structure of the stent of FIG. 6A when crimped;

FIG. 6C depicts the structure of the stent of FIG. 6B when expanded;

FIG. 7A is an elevation of a stent according to an embodiment of the invention crimped in a longitudinally asymmetric position around a balloon;

FIG. 7B is an elevation of the stent and balloon of FIG. 7B expanded with the balloon;

FIG. 8A is an elevation of a stent according to an embodiment of the invention crimped in around a conical balloon;

FIG. 8B is an elevation of the stent and balloon of FIG. 8A expanded;

FIG. 9A is a longitudinal cross-section of a standard monorail angioplasty balloon catheter;

FIG. 9B is a longitudinal cross-section of a monorail angioplasty balloon catheter according to an embodiment of the invention;

FIG. 10A is a longitudinal cross-section of a monorail angioplasty balloon catheter according to another embodiment of the invention;

FIG. 10B is a longitudinal cross-section of a monorail angioplasty balloon catheter according to another embodiment of the invention;

FIG. 11A is a longitudinal cross-section of a standard monorail angioplasty balloon catheter;

FIG. 11B is a longitudinal cross-section of a monorail angioplasty balloon catheter according to an embodiment of the invention;

FIG. 12A is a diagrammatic elevation view of a standard monorail angioplasty balloon catheter;

FIG. 12B is a diagrammatic elevation view of a monorail angioplasty balloon catheter according to an embodiment of the invention;

FIG. 12C is a diagrammatic elevation view of a standard intracranial catheter;

FIG. 13 is a diagrammatic view of a monorail angioplasty balloon catheter and stent during a stage of emplacement in an intracranial artery of a patient; and

FIG. 14 is a diagrammatic view of a monorail angioplasty balloon catheter and stent during a later stage of emplacement in an intracranial artery of a patent.

While various embodiments are amenable to various modifications and alternative forms, specifics thereof have been shown by way of example in the drawings and will be described in detail. It should be understood, however, that the intention is not to limit the claimed inventions to the particular embodiments described. On the contrary, the intention is to cover all modifications, equivalents, and alternatives falling within the spirit and scope of the subject matter as defined by the claims.

### DETAILED DESCRIPTION OF THE DRAWINGS

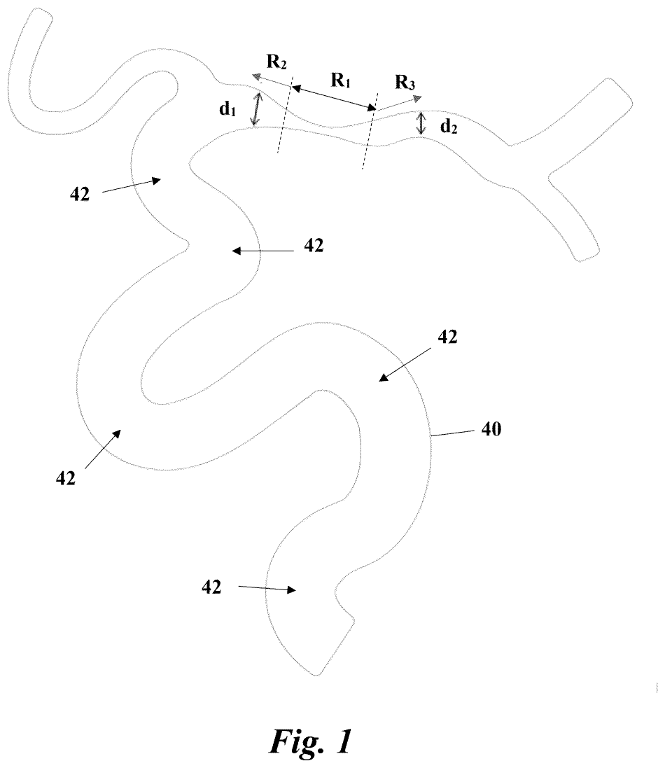

FIG. 1 is a diagrammatic depiction of an intracranial artery 40. Such arteries 40 typically have many tortuous bends 42, and have walls that are much thinner than arteries in other areas of the body, making treatment with stents delivered by catheters more difficult to accomplish. A region R1 of the depicted artery 40 exhibits stenosis—that is, an abnormal narrowing. The region R2 on the proximal side of the stenotic region R1 will often have a greater diameter d1 than the diameter d2 of region R3 on the distal side of region R1.

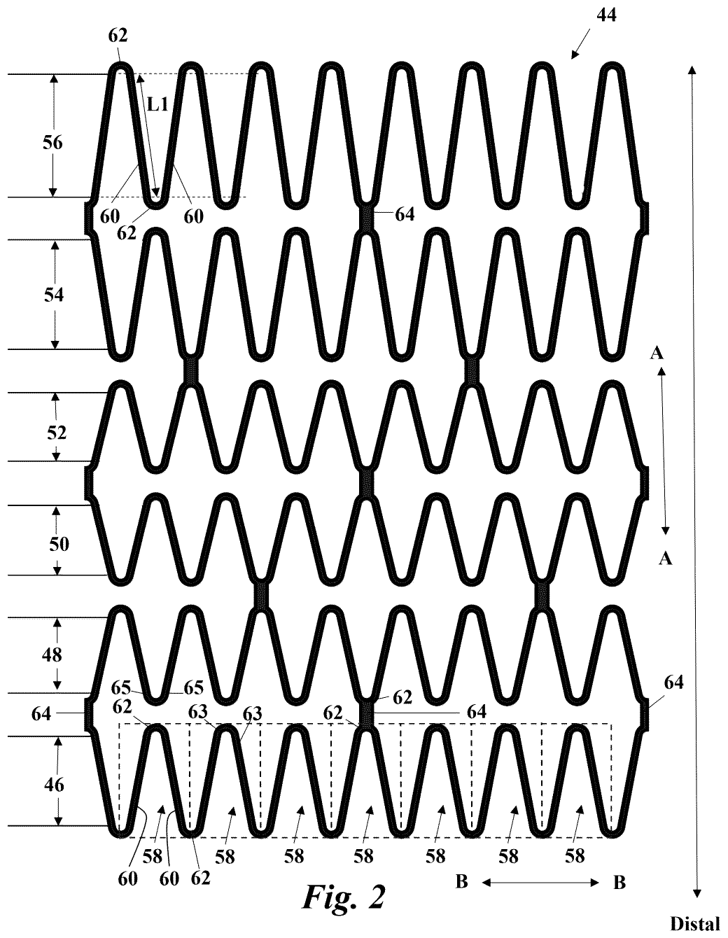

A stent is a cylindrical fenestrated mass like structure that is deployed in an artery to increase open area available for blood flow through a region narrowed by stenosis. FIG. 2 is a diagram depicting the structure of an open-cell (wall fenestration area greater than solid area) intracranial stent 44, in a flat state before being formed into a cylinder, according to embodiments of the invention. Stent 44 generally includes a plurality of rings 46, 48, 50, 52, 54, 56, progressing sequentially from a distal end to a proximal end along longitudinal direction A-A. Each of rings 46, 48, 50, 52, 54, 56, has a sinusoidal configuration as depicted, and can be laser cut from cobalt chromium alloy, stainless steel, nickel and cobalt free alloy such as Vasculoy® available from MeKo Manufacturing e.K. of Sarstedt, Germany, or from any other material suitable for use within an artery. Cells 58 are arranged adjacently in each of rings 46, 48, 50, 52, 54, 56, in a transverse direction B-B. Each cell 58 of the respective ring in which it is arranged can be characterized as having struts 60, and peaks 62 where ends 63 of struts 60 join. Peaks are also formed where opposing ends 65 are joined in adjacent cells. Peaks 62 of adjacent rings (for example ring 46 and ring 48) are arranged directly opposing as depicted, and are joined together by a nodal connection 64 at about every 3^{rd} or 4^{th} adjacent peak. Although six rings 46, 48, 50, 52, 54, 56, are depicted, it will be appreciated that the number of rings may be made fewer or more while remaining within the scope of the invention.

The size of each cell 58 in the respective ring 46, 48, 50, 52, 54, 56, is influenced primarily by strut length dimension L1. As depicted, cell 58 varies in size amongst rings 46, 48, 50, 52, 54, 56. If distal most ring 46 (the ring which will be deployed furthest distally in the artery) is considered the “reference” ring for strut length L1 (thus having a relative value of 1.0), it has been found to be efficacious if ring 48 has a relative value for strut length L1 of about 0.9, rings 50 and 52 about 0.85, ring 54 about 1.15, and ring 56 about 1.25. It will be appreciated that each of these relative values may be varied slightly (e.g. +0.05) while remaining within the scope of the invention. These proportions of cell 58 size enable stent 44 to be conformably shaped during emplacement in a typical intracranial artery 40 with a greater diameter d₁ proximal to the stenotic region R₁ than the diameter d₂ on the distal side of the stenosis. In embodiments of the invention, struts 60 will have a thickness of from about 40 microns to about 60 microns after being laser cut from a sheet of material and polished. This enables the stent to be deployed at low pressure inflation of an underlying balloon at 4-6 atmospheres.

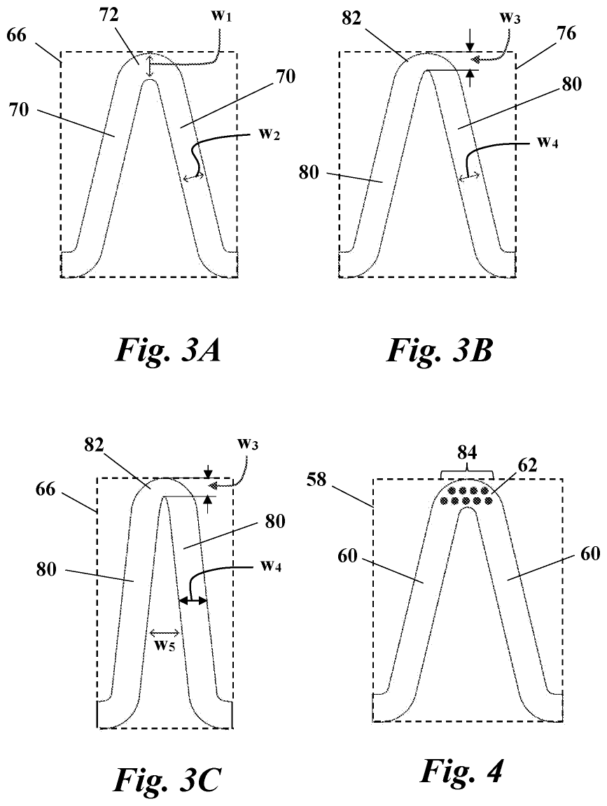

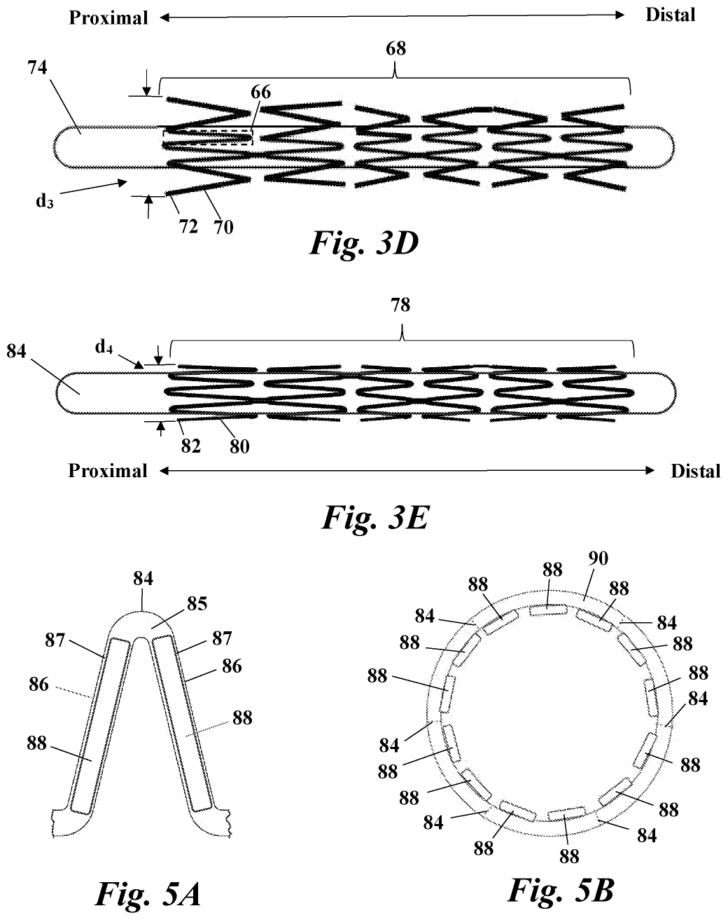

Another feature of embodiments of the invention is depicted in FIGS. 3A-3E. In FIGS. 3A and 3D, an exemplary cell 66 of a stent 68 is depicted. Notably, a width w₁ at peak 72 is approximately equal to a width w₂ along strut 70. An exemplary cell 76 of a stent 78 according to another embodiment of the invention is depicted in FIGS. 3b, 3c, and 3e. As depicted, width w₃ at peak 82 is made substantially less than width w₄ along strut 80. The effect of this reduced width w₃ at peak 82 is depicted in FIG. 3C, with the stent 78 crimped as it will be prior to deployment. The reduced width w₃ at peak 82 enables width w₅ between struts 80 to be less than would be possible with the configuration of FIG. 3A. This enables stent 78 to be crimped into a tighter cylinder for improved ability to deploy the stent. For example, FIG. 3D shows stent 68 with the exemplary cell 66 of FIG. 3A, wherein the stent 68 is crimped around a balloon 74. The stent 68 has the same ring configuration and relative strut dimensions amongst rings of FIG. 2. Peaks 72 distort outward, leading to a greatest cylinder diameter of d₃. In FIG. 3E, stent 78 is crimped around a balloon 84. Again, the stent 78 has the same ring configuration and relative strut dimensions amongst rings of FIG. 2. Due to the reduced width w₃ at peak 82 relative to width w₄ of strut 80, peaks 82 distort outward to a lesser degree, leading to a greatest cylinder diameter of d₄, which is less than diameter d₃. This is advantageous, as it reduces the profile of the stent 78 for easier insertion and deployment of the stent.

Another feature of embodiments of the invention is depicted in FIG. 4. In this embodiment, microholes 84 can be provided and filled with a non-toxic radiopaque substance such as Tantalum (Ta) or Tantalum Oxide (Ta₂O₅) to enable visualization of the position of the stent via radiographic imaging during insertion and deployment.

Yet another feature of embodiments of the invention is depicted in FIGS. 5A and 5B. As depicted in FIG. 5A, inside surface 87 of stent struts 86 can have a layer 88 of a mixture of nanoparticles of an anti-thrombotic medication (such as Tirofiban 1-4 mg) and a bioabsorbable polymer such as polyanhydride. The anti-thrombotic medication nanoparticles can be prepared using water-in-oil (W/O), oil-in-water (O/W), or double emulsion (W/O/W) methods as known in the art. The nanoparticles will be mixed with the polyanhydride using a solvent evaporation technique followed by evaporation and subsequent dehydration. Stent will be coated using “dip coating” by submerging the stent in the solution of a solvent followed by evaporation in ambient air or an oven. The controlled, nonenzymatic degradation of the polyanhydrides will release the anti-thrombotic medication within 24 hours in the patient and reduce thrombogenicity of the stent. This can reduce local thrombosis in patients who have not received antithrombotic medication prior to the emplacement procedure, such as in acute ischemic stroke. As depicted in the end view of FIG. 5B, these layers 88 will face into the interior of the stent 90 when deployed.

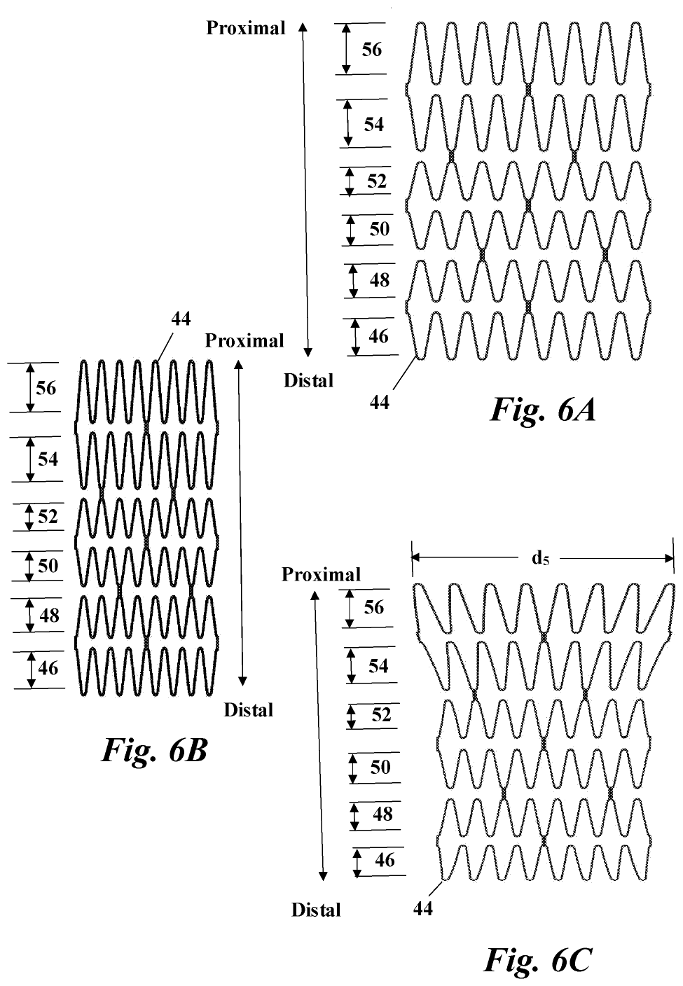

FIGS. 6A-6C depict the expansion of stent 44 when expanded using a balloon (not depicted). Stent 44 has the ring 46, 48, 50, 52, 54, 56, configuration and relative dimensions of FIG. 2. In FIG. 6A, stent 44 is depicted before crimping, and FIG. 6B depicts the crimped stent 44. In FIG. 6C, stent 44 has been expanded using a cylindrical balloon (not depicted). Stent 44 assumes a conical or hyperboloid shape as depicted, conforming to artery wall 40 when deployed in a stenotic region R₁. Notably, proximal-most ring 46 expands to greatest cylinder diameter of d₅, and rings 48, 50, 52, 54, 56, can expand to a degree proportional to the 1.25, 1.15, 0.85, 0.85, 0.9, and 1.0 respective ratios, proceeding from proximal to distal along the stent 44.

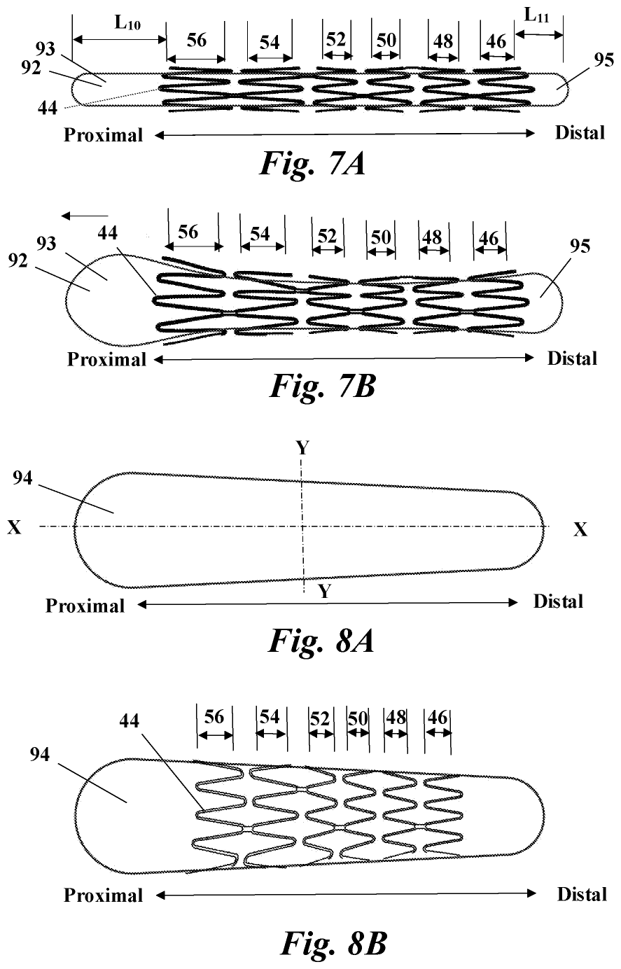

FIG. 7A depicts stent 44 of FIG. 2 crimped around a cylindrical balloon 92 prior to deployment. As depicted in FIG. 7B, the novel configuration of rings 46, 48, 50, 52, 54, 56, enables stent 44 to assume a conical or hyperboloid shape when balloon 92 is inflated, enabling stent 44 to conform to the walls of an artery 40 when deployed in a stenotic region R₁. It will be appreciated that stent 44 is initially crimped over balloon 92 asymmetrically lengthwise, with length L₁₀ being 2 to 3 times length L₁₁. This facilitates greater expansion of balloon 92 at proximal end 93 as opposed to distal end 95, leading to a generally conical or hyperboloid shape of stent 44 when balloon is inflated as in FIG. 7B.

An alternative embodiment is depicted in FIGS. 8A-8C. Balloon 94 assumes a generally conical shape when inflated as depicted in FIG. 8A. Balloon 94 can be formed by blow molding in a conical mold (not depicted) where heated jaws (not depicted) forming the walls of the mold are not parallel but inclined in relation to each other creating a conical tunnel in which balloon 94 is molded. When stent 44 is crimped around balloon 94 and balloon 94 is inflated, stent 44 will also assume a conical shape as depicted in FIG. 8B, enabling stent 44 to conform to the walls of an artery 40 when deployed in a stenotic region R₁. It is desirable if balloon 94 underlying stent 44 inflates asymmetrically, with a 0.25-0.5 mm reduction in diameter in the direction of lateral axis Y-Y per 10 mm distance along longitudinal axis X-X, proceeding from proximal to distal. The larger inflation proximally enables greater expansion of stent 44 in the proximal portion relative to the distal portion.

Embodiments of the invention also include apparatuses and methods for placing the innovative stent described herein in patients with intracranial stenosis to reduce the risk of ischemic stroke. The delivery apparatus is built on the platform of a standard monorail angioplasty balloon catheter and balloon expandable stent platform to ensure that any additional insertion of new devices is not necessary. The apparatus is introduced through the femoral or radial artery through standard guide catheters of sheaths which are already placed in the internal carotid artery or vertebral artery. The apparatus is introduced over a standard microwire into the intracranial arteries. A longer shaft allows delivery of the stent through intracranial catheters that have a working length of 130-135 cm. The ultrathin open cell design of the stent reduces the delivery profile and increases the flexibility of the mounted stent prior to, and after, deployment. As described elsewhere herein, the delivery profile may be further reduced by the smaller diameter of the struts at the end of the peak enabling greater reduction of the angle of the peak during crimping and reducing the profile of the device This enables the stent to traverse the tortuous intracranial arteries prior to reaching the target lesion. As will be described in further detail below, embodiments of the apparatus increase trackability by using hydrophilic coatings and longer length between the distal tip and the exit port for the microwire. The length between the distal tip and the exit port for the microwire of this novel system enables a larger segment in the monorail angioplasty balloon catheter than existing systems, in which flexibility can be modulated by placement of stiff or less stiff components of the microwire through the segment. Once the stent is in position, the stent can be deployed by inflation of the underlying balloon. The larger cell sizes proximally allow greater expansion of the stent proximal to the stenosis compared with distal to the stenosis, leading to a conical or hyperboloid configuration of the stent to match the arterial wall. Furthermore, the stent can deployed in an asymmetrical manner (larger diameter proximally) due to larger inflation of the balloon because of a larger proximal segment of balloon not constrained by the overlying crimped stent.

Generally, a monorail angioplasty balloon catheter is an angioplasty balloon catheter in which the microwire passes through the balloon itself, entering the balloon portion from the distal most portion, exiting the catheter proximal to the balloon, and running parallel to a shaft through a guide catheter. The system has a single lumen shaft (for inflation of the balloon) which runs from proximal most portion to the balloon, and a second short central lumen within the distal balloon portion. The distal lumen is advanced over the microwire.

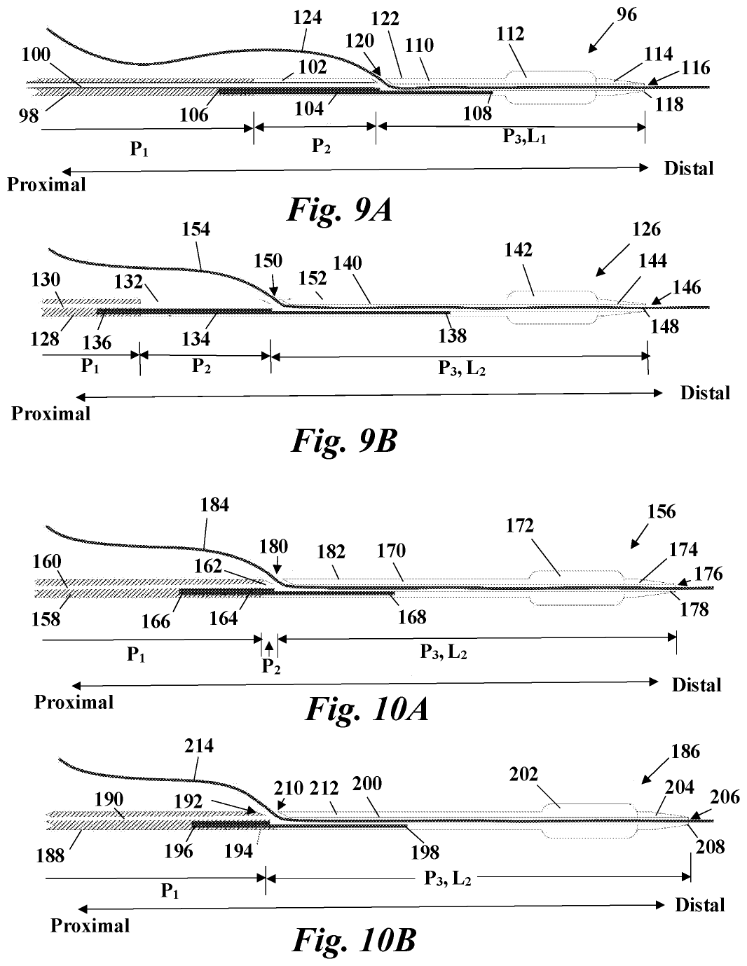

As depicted in FIG. 9A, a standard monorail angioplasty balloon catheter 96 for stent delivery has three segments. Segment P₁ generally includes a relatively rigid hypodermic tube (“hypotube”) shaft 98 (also known as a “pusher”) with lumen 100. Segment P₁ can be made from high polymer and/or metallic materials. Central transition segment P₂ merges with segment P₁ and has polymer body 102 with continuing central lumen 100, and tapered mandrel 104. Tapered mandrel 104 tapers gradually from proximal end 106 to distal end 108, and can be covered with polytetrafluoroethylene (PTFE) or another polymer to improve lubricity. Distal segment P₃ generally includes body portion 110, balloon 112, and tip portion 114. Body portion 110 defines two lumens: continuing central lumen 100 which opens into balloon 112 enabling introduction of air to inflate balloon 112, and second lumen 116. Second lumen 116 extends from distal tip 118 and terminates in adjacent central transition segment P₂ with fenestration 120 through exterior wall 122 of body portion 110. Microwire 124 extends through second lumen 116 from distal tip 118 and out through fenestration 120 so that monorail angioplasty balloon catheter 96 is slidable thereon. Balloon 112 can be made various sizes as suitable for specific applications, and is sealingly bonded at proximal and distal ends on distal segment P₃. Balloon 112 can be made from (is but not limited to) thermoplastic polymer, polyethylene terephthalate (PET), nylon, polyethylene with or without polyvinyl chloride (PVC), Pebax®, or polyurethane. Cone shaped tip portion 114 is the distal most portion beyond balloon 112, and serves as the first portion of monorail angioplasty balloon catheter 96 to traverse a stenosis.

A monorail angioplasty balloon catheter 126 according to embodiments of the invention is depicted in FIG. 9B. Again, segment P₁ generally includes a relatively rigid hypotube shaft 128 with central lumen 130. Segment P₁ can be made from high polymer and/or metallic materials. Central transition segment P₂ merges with segment P₁ and generally includes hypotube 132 with continuing central lumen 130, and tapered mandrel 134. Tapered mandrel 134 tapers gradually from proximal end 136 to distal end 138, and can be covered with PTFE or another polymer to improve lubricity. Distal segment P₃ generally includes body portion 140, balloon 142, and tip portion 144. Body portion 140 defines two lumens: continuing central lumen 130 which opens into balloon 142 enabling introduction of air to inflate balloon 142, and second lumen 146. Second lumen 146 extends from distal tip 148 and terminates in adjacent central transition segment P₂ with fenestration 150 through exterior wall 152 of body portion 140. Microwire 154 extends through second lumen 146 from distal tip 148 and out through fenestration 150 so that monorail angioplasty balloon catheter 126 is slidable thereon. The inner surface of second lumen 146 may be coated with a hydrophilic coating which may be (but is not limited to) polyvinylpyrrolidone (PVP); polyurethane; polyacrylic acid; polyethylene oxide; polysaccharide and/or cross-linked polymer to reduce friction and facilitate sliding of monorail angioplasty balloon catheter 126 over microwire 154. Balloon 142 can be made various sizes as suitable for specific applications, and is sealingly bonded at proximal and distal ends on distal segment P₃. Balloon 142 can be made from (but is not limited to) thermoplastic polymer, PET, nylon, polyethylene with or without PVC, Pebax®, or polyurethane. Cone shaped tip portion 144 is the distal most portion beyond balloon 142, and again serves as the first portion of monorail angioplasty balloon catheter 126 to traverse a stenosis.

Monorail angioplasty balloon catheter 126 of FIG. 9B differs from monorail angioplasty balloon catheter 96 of FIG. 9A, in that the length of distal segment P₃ is increased in monorail angioplasty balloon catheter 126. Generally, in the standard monorail angioplasty balloon catheter 96 of FIG. 9A, distal segment P₃ will have a longitudinal length dimension L₁ of <25 cm. In contrast, monorail angioplasty balloon catheter 126 of FIG. 9B has a longitudinal length dimension L₂ of 30-40 cm. This increased length of distal segment P₃ in monorail angioplasty balloon catheter 126 enables a relatively longer segment of microwire 154 in distal segment P₃, enabling greater trackability and advancement of the device through longer intracranial catheters.

Another embodiment of a monorail angioplasty balloon catheter 156 is depicted in FIG. 10A. Segment P₁ generally includes a relatively rigid hypotube shaft 158 with central lumen 160. Segment P₁ can be made from high polymer and/or metallic materials. Central transition segment P₂ merges with segment P₁ and generally includes hypotube 162 with continuing central lumen 160, and tapered mandrel 164. Tapered mandrel 164 tapers gradually from proximal end 166 to distal end 168, and can be covered with PTFE or another polymer to improve lubricity. Distal segment P₃ generally includes body portion 170, balloon 172, and tip portion 174. Body portion 170 defines two lumens: continuing central lumen 160 which opens into balloon 172 enabling introduction of air to inflate balloon 172, and second lumen 176. Second lumen 176 extends from distal tip 178 and terminates in adjacent central transition segment P₂ with fenestration 180 through exterior wall 182 of body portion 170. Microwire 184 extends through second lumen 176 from distal tip 178 and out through fenestration 180 so that monorail angioplasty balloon catheter 156 is slidable thereon. The inner surface of second lumen 176 may be coated with a hydrophilic coating which may be (but is not limited to) PVP; polyurethane; polyacrylic acid; polyethylene oxide; polysaccharide and/or cross-linked polymer to reduce friction and facilitate sliding of monorail angioplasty balloon catheter 156 over microwire 184. Again, balloon 172 can be made various sizes as suitable for specific applications, is sealingly bonded at proximal and distal ends on distal segment P₃, and can be made from (but is not limited to) thermoplastic polymer, PET, nylon, polyethylene with or without PVC, Pebax®, or polyurethane. Cone shaped tip portion 174 is the distal most portion beyond balloon 172, and again serves as the first portion of monorail angioplasty balloon catheter 156 to traverse a stenosis.

Monorail angioplasty balloon catheter 156 of FIG. 10A differs from monorail angioplasty balloon catheter 126 of FIG. 9B, in that the length of transition segment P₂ is decreased to ≤ 7 mm. This reduces the distance between the distal most portion of segment P₁ (or “pusher”) and fenestration 180 through which microwire 184 exits in segment P₃.

Another embodiment of a monorail angioplasty balloon catheter 186 is depicted in FIG. 10B. Segment P₁ again generally includes a relatively rigid hypotube shaft 188 with central lumen 190. Segment P₁ can be made from high polymer and/or metallic materials. In comparison with the embodiment of FIG. 10A, central transition segment P₂ is eliminated entirely, and is replaced by tapered end 192 of hypotube shaft 188, which can be formed by laser cutting. Tapered mandrel 194 tapers gradually from proximal end 196 to distal end 198, and can be covered with PTFE or another polymer to improve lubricity. Distal segment P₃ generally includes body portion 200, balloon 202, and tip portion 204. Body portion 200 defines two lumens: continuing central lumen 190 which opens into balloon 202 enabling introduction of air to inflate balloon 202, and second lumen 206. Second lumen 206 extends from distal tip 208 and terminates adjacent fenestration 210 through exterior wall 212 of body portion 200. Microwire 214 extends through second lumen 206 from distal tip 208 and out through fenestration 210 so that monorail angioplasty balloon catheter 186 is slidable thereon. The relief provided by tapered end 192 of hypotube shaft 188 adjacent fenestration 210 enables microwire 214 to exit fenestration 210 smoothly and without undue binding. The inner surface of second lumen 206 may be coated with a hydrophilic coating which may be (but is not limited to) PVP; polyurethane; polyacrylic acid; polyethylene oxide; polysaccharide and/or cross-linked polymer to reduce friction and facilitate sliding of monorail angioplasty balloon catheter 196 over microwire 214. Again, balloon 202 can be made various sizes as suitable for specific applications, is sealingly bonded at proximal and distal ends on distal segment P₃, and can be made from (but is not limited to) thermoplastic polymer, PET, nylon, polyethylene with or without PVC, Pebax®, or polyurethane. Cone shaped tip portion 204 is the distal most portion beyond balloon 202, and again serves as the first portion of monorail angioplasty balloon catheter 196 to traverse a stenosis.

The modified segment P₃ of monorail angioplasty balloon catheters, 126, 156, 186, with increase in length (30-40 cm) between the distal tip and the exit port for the microwire, providing a longer segment of microwire in the catheter compared with a standard monorail angioplasty balloon catheter provides greater trackability and advancement of the device through longer intracranial catheters. In addition, the overall stiffness of the monorail angioplasty balloon catheter can be more effectively modified through use of microwire with segments having greater or lesser stiffness.

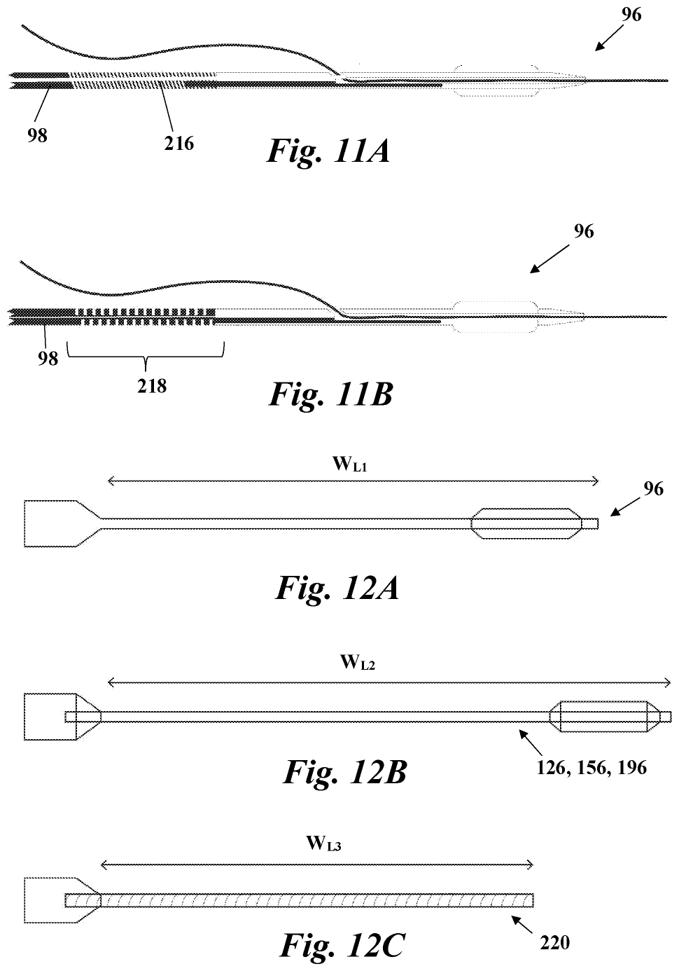

Another aspect of embodiments of the invention is depicted in FIGS. 11A and 11B. FIG. 11A depicts a standard monorail angioplasty balloon catheter 96 such as depicted in FIG. 7A. Coiled segment 216 is coupled with hypotube shaft 98 to improve flexibility. In the embodiment of FIG. 11B, hypotube shaft 98 is provided with exterior grooving 218 to improve flexibility in place of coiled segment 216. Alternatively, hypotube shaft 98 can be provided with multiple closely spaced fenestrations to the same effect.

Another aspect of embodiments of the invention is depicted in FIGS. 12A-C. Working length refers to the total length of a monorail angioplasty balloon catheter which can be inserted in other catheters (such as guide catheters), and beyond, into the intracranial arteries. FIG. 12A generally depicts a standard monorail angioplasty balloon catheter 96 in which working length W_{L1} is about 138-142 cm. FIG. 12B depicts monorail angioplasty balloon catheters 126, 156, 186, in which working length W_{L2} is about 145-155 cm. This increase in working length enables monorail angioplasty balloon catheters 126, 156, 186, to be more effectively employed to access a stenosis in a remote, tortuous, region of an intracranial artery.

The relatively large distal segment P₃ of monorail angioplasty balloon catheters 126, 156, 186, provides for the stiffness to be modified by inserting a stiff or more flexible portion of a microwire in the lumen of monorail angioplasty balloon catheter. FIG. 12C depicts a standard intracranial catheter 220 through which the monorail angioplasty balloon catheters 126, 156, 186, can be delivered. Standard intracranial catheter 220 will generally have an interior lumen with a diameter ranging from 4-8 F (French). The intracranial catheter 220 will typically have been previously placed in the patient's arteries though a standard guide catheter or guide sheath. The longer working length W_{L2} of monorail angioplasty balloon catheters 126, 156, 186, ensures that a stent mounted on the balloon delivery system can be inserted through the previously placed commercially available intracranial catheter 220, which typically has a working length W_{L3} of 130-135 cm. and ensures that the monorail angioplasty balloon catheters 126, 156, 186, and stent mounted thereon can exit through the intracranial catheter 220 and be placed across the stenosis without being constrained in the lumen of the previously placed intracranial catheter 220.

As detailed above, stents 44, 68, 78, 90 are delivered through a modified monorail angioplasty balloon catheter 126, 156, 186. The monorail angioplasty balloon catheter 126, 156, 186 has a working length W_{L2} of about 145-155 cm (compared to a working length W_{L1} of about 138-142 cm for a standard monorail angioplasty balloon catheter 96), and the exit port 150, 180, 210, for microwire 154, 184, 214, is located 35-55 cm from the catheter tip 148, 178, 208, which is longer than the standard monorail angioplasty balloon catheter 96 with exit port 120 located 25-35 cm from catheter tip 118.

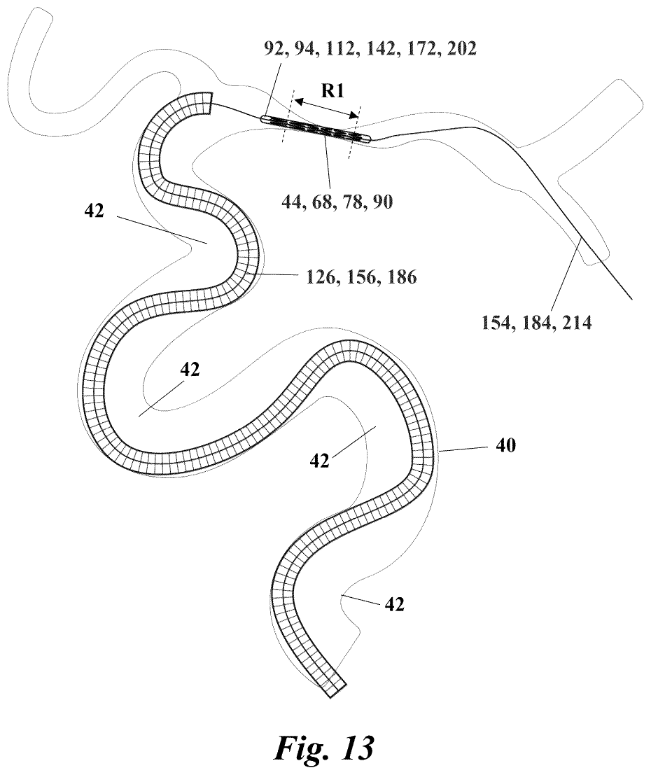

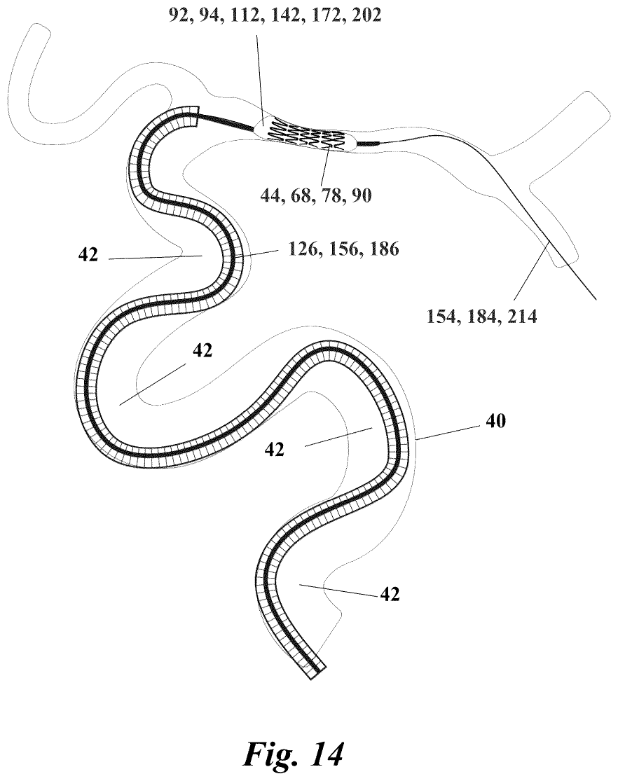

FIG. 13 depicts schematically the deployment of stent 44, 68, 78, 90, in an intracranial artery 40 with tortuous bends 42 in a patient. Stent 44, 68, 78, 90, is first crimped around balloon 92, 94, 112, 142, 172, 202. Then monorail angioplasty balloon catheter 126, 156, 186, is advanced over microwire 154, 184, 214, through artery 40 until stent 44, 68, 78, 90, is positioned across stenotic region R₁. With stent 44, 68, 78, 90, in position, balloon 92, 94, 112, 142, 172, 202, can be inflated as depicted in FIG. 14 to expand the stent. It will be appreciated that balloon inflation can be performed iteratively to ensure the proximal end stent 44, 68, 78, 90, is properly conformed to the artery wall. For example, balloon 92, 94, 112, 142, 172, 202, can be inflated in a distal-most position to initially expand stent 44, 68, 78, 90, the balloon deflated, then the monorail angioplasty balloon catheter with attached balloon withdrawn to a more proximal position and reinflated to further expand the proximal portion of stent 44, 68, 78, 90. This process can be repeated as many times as needed to conform the stent.

Various embodiments of systems, devices, and methods have been described herein. These embodiments are given only by way of example and are not intended to limit the scope of the claimed inventions. It should be appreciated, moreover, that the various features of the embodiments that have been described may be combined in various ways to produce numerous additional embodiments. Moreover, while various materials, dimensions, shapes, configurations and locations, etc. have been described for use with disclosed embodiments, others besides those disclosed may be utilized without exceeding the scope of the claimed inventions.

Persons of ordinary skill in the relevant arts will recognize that the subject matter hereof may comprise fewer features than illustrated in any individual embodiment described above. The embodiments described herein are not meant to be an exhaustive presentation of the ways in which the various features of the subject matter hereof may be combined. Accordingly, the embodiments are not mutually exclusive combinations of features; rather, the various embodiments can comprise a combination of different individual features selected from different individual embodiments, as understood by persons of ordinary skill in the art. Moreover, elements described with respect to one embodiment can be implemented in other embodiments even when not described in such embodiments unless otherwise noted.

Although a dependent claim may refer in the claims to a specific combination with one or more other claims, other embodiments can also include a combination of the dependent claim with the subject matter of each other dependent claim or a combination of one or more features with other dependent or independent claims. Such combinations are proposed herein unless it is stated that a specific combination is not intended.

Any incorporation by reference of documents above is limited such that no subject matter is incorporated that is contrary to the explicit disclosure herein. Any incorporation by reference of documents above is further limited such that no claims included in the documents are incorporated by reference herein. Any incorporation by reference of documents above is yet further limited such that any definitions provided in the documents are not incorporated by reference herein unless expressly included herein.

For purposes of interpreting the claims, it is expressly intended that the provisions of 35 U.S.C. § 112(f) are not to be invoked unless the specific terms “means for” or “step for” are recited in a claim.

What is claimed is:

1. An open cell intracranial stent comprising:
a plurality of rings arranged sequentially in a longitudinal direction, each ring comprising a plurality of cells arranged adjacently to each other in a transverse direction generally perpendicular to the longitudinal direction, each cell comprising a pair of struts, each strut in the cell having a first end and an opposing second end, the first ends of the pair of struts joined at a first peak, the second end of each of the pair of struts joined to a second end of one of the pair of struts in a separate, adjacent, one of the plurality of cells in the ring to form a pair of peaks opposing the first peaks, such that the struts and peaks in the ring form a generally sinusoidal configuration, each of the plurality of rings being joined to another one of the plurality of rings at a nodal connection, each of the pair of struts in each one of the plurality of cells in each ring having a uniform strut length dimension; and
wherein the stent has a pair of opposing ends and the plurality of rings includes a first ring at one of the opposing ends, a second ring, a third ring, a fourth ring, a fifth ring, and a sixth ring at the other of the opposing ends, the uniform strut length dimension of the second ring cells having a relative value of 0.9 times the uniform strut length dimension of the cells of the first ring, the third ring cells having a relative value of 0.85 times the uniform strut length dimension of the cells of the first ring, the fourth ring cells having a relative value of 0.85 times the uniform strut length dimension of the cells of the first ring, the fifth ring cells having a relative value of 1.15 times the uniform strut length dimension of the cells of the first ring, and the sixth ring cells having a relative value of 1.25 times the uniform strut length dimension of the cells of the first ring.

2. The stent of claim 1, wherein each of the struts is coated with a layer comprising a mixture of nanoparticles of an anti-thrombotic medication and a bioabsorbable polymer.

3. The stent of claim 2, wherein the anti-thrombotic medication is Tirofiban and the bioabsorbable polymer is polyanhydride.

4. The stent of claim 1, wherein the stent defines a plurality of microholes, the microholes filled with a radiopaque substance.

5. The stent of claim 4, wherein the radiopaque substance is Tantalum or Tantalum Oxide.

6. The stent of claim 1, wherein the struts have a thickness of from about 40 microns to about 60 microns.

7. The stent of claim 1, wherein a width at the first peak and a width at each of the second peaks is less than a width of the struts.

8. The stent of claim 1, wherein the stent forms a conical or hyperboloid shape when expanded.

9. A monorail angioplasty balloon catheter comprising:
a first segment comprising a first hypotube tube shaft defining a first lumen;
a second segment operably coupled to the first segment and comprising a first body portion defining a second lumen fluidly coupled to the first lumen, and a tapered mandrel;
a third segment operably coupled to the second segment and comprising a second body portion, a balloon, and a tip portion, the body portion defining a third lumen fluidly coupling the first and second lumen and the balloon, and a fourth lumen, the fourth lumen adapted to receive a microwire therethrough and extending from an end of the tip portion to a fenestration extending through an outer wall of the body portion adjacent the second segment;
wherein a longitudinal length dimension of the third segment is from about 30 cm to about 40 cm.

10. The monorail angioplasty balloon catheter of claim 9, wherein a working length of the monorail angioplasty balloon catheter is from about 145 cm to about 155 cm.

11. The monorail angioplasty balloon catheter of claim 9, wherein an interior surface of the fourth lumen is coated with a hydrophilic coating selected from the group consisting of PVP, polyurethane, polyacrylic acid, polyethylene oxide, polysaccharide and cross-linked polymer.

12. The monorail angioplasty balloon catheter of claim 9, wherein the tapered mandrel is coated with PTFE or another polymer.

13. The monorail angioplasty balloon catheter of claim 9, wherein the balloon is formed from a material selected from the group consisting of thermoplastic polymer, PET, nylon, polyethylene with or without PVC, Pebax®, and polyurethane.

14. A monorail angioplasty balloon catheter comprising:
a first segment comprising a first hypotube tube shaft defining a first lumen;
a second segment operably coupled to the first segment and comprising a body portion, a balloon, and a tip portion, the body portion defining a second lumen fluidly coupling the first lumen and the balloon, and a third lumen, the third lumen adapted to receive a microwire therethrough and extending from an end of the tip portion to a fenestration extending through an outer wall of the body portion adjacent the first segment, an end of the first segment being tapered adjacent the fenestration;
wherein a longitudinal length dimension of the second segment is from about 30 cm to about 40 cm.

15. The monorail angioplasty balloon catheter of claim 14, wherein a working length of the monorail angioplasty balloon catheter is from about 145 cm to about 155 cm.

16. The monorail angioplasty balloon catheter of claim 14, further comprising a tapered mandrel.

17. The monorail angioplasty balloon catheter of claim 14, wherein an interior surface of the third lumen is coated with a hydrophilic coating selected from the group consisting of PVP, polyurethane, polyacrylic acid, polyethylene oxide, polysaccharide and cross-linked polymer.

18. The monorail angioplasty balloon catheter of claim 14, wherein the balloon is formed from a material selected from the group consisting of thermoplastic polymer, PET, nylon, polyethylene with or without PVC, Pebax®, and polyurethane.

19. A method of deploying an intracranial stent in a stenosis in an intracranial artery of a patient, the method comprising:
providing an intracranial stent comprising:
a plurality of rings arranged sequentially in a longitudinal direction, each ring comprising a plurality of cells arranged adjacently to each other in a transverse direction generally perpendicular to the longitudinal direction, each cell comprising a pair of struts, each strut in the cell having a first end and an opposing second end, the first ends of the pair of struts joined at a first peak, the second end of each of the pair of struts joined to a second end of one of the pair of struts in a separate, adjacent, one of the plurality of cells in the ring to form a pair of peaks opposing the first peaks, such that the struts and peaks in the ring form a generally sinusoidal configuration, each of the plurality of rings being joined to another one of the plurality of rings at a nodal connection, each of the pair of struts in each one of the plurality of cells in each ring having a uniform strut length dimension; and
wherein the stent has a pair of opposing ends and the plurality of rings includes a first ring at one of the opposing ends, a second ring, a third ring, a fourth ring, a fifth ring, and a sixth ring at the other of the opposing ends, the uniform strut length dimension of the second ring cells having a relative value of 0.9 times the uniform strut length dimension of the cells of the first ring, the third ring cells having a relative value of 0.85 times the uniform strut length dimension of the cells of the first ring, the fourth ring cells having a relative value of 0.85 times the uniform strut length dimension of the cells of the first ring, the fifth ring cells having a relative value of 1.15 times the uniform strut length dimension of the cells of the first ring, and the sixth ring cells having a relative value of 1.25 times the uniform strut length dimension of the cells of the first ring;
providing a monorail angioplasty balloon catheter comprising:
a first segment comprising a first hypotube tube shaft defining a first lumen;
a second segment operably coupled to the first segment and comprising a first body portion defining a second lumen fluidly coupled to the first lumen, and a tapered mandrel;
a third segment operably coupled to the second segment and comprising a second body portion, a balloon, and a tip portion, the body portion defining a third lumen fluidly coupling the first and second lumen and the balloon, and a fourth lumen, the fourth lumen adapted to receive a microwire therethrough and extending from an end of the tip portion to a fenestration extending through an outer wall of the body portion adjacent the second segment;
wherein a longitudinal length dimension of the third segment is from about 30 cm to about 40 cm;
crimping the stent around the balloon of the catheter;
introducing the monorail angioplasty balloon catheter into the intracranial artery;
positioning the stent across the stenosis; and
inflating the balloon to expand the stent.

20. The method of claim 19, further comprising deflating the balloon, repositioning the balloon proximally relative to the stent, and reinflating the balloon.
